(19) United States
(12) Patent Application Publication
(10) Pub. No.: US 2007/0237802 A1
(43) Pub. Date: Oct. 11, 2007

(54) Inhibition of Calcification on an Endovascular Device

(75) Inventor: William F. McKay, Memphis, TN (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., Santa Rosa, CA (US)

(21) Appl. No.: 11/279,325

(22) Filed: Apr. 11, 2006

(51) Int. Cl. A61F 2/06; A61K 38/19; A61K 38/18

(52) U.S. Cl. 424/423; 514/2; 623/1.48; 514/12; 424/426; 424/85.1

(57) ABSTRACT

An endovascular device includes a body having a surface, and at least one protein or peptide antagonist of calcification disposed on a portion of the surface. In another embodiment of the invention, an endoluminal device includes a body having a surface, and a coating disposed on a portion of the surface. The coating includes a transforming growth factor beta receptor antagonist. Another embodiment of the invention provides a method of inhibiting calcification of an endoluminal device. The method includes providing a body including a surface, disposing at least one protein or peptide antagonist of calcification on a portion of the surface, and deploying the body at a treatment site.

### TECHNICAL FIELD

This invention relates generally to endovascular medical devices, and particularly to the inhibition of calcification on the same.

### BACKGROUND OF THE INVENTION

Numerous endovascular devices have been developed for the treatment of a variety of cardiovascular pathologies. For example, endovascular valve prostheses have been developed for pulmonary valve stenosis. The disorder commonly results from a congenital defect, and is present at birth, but is also associated with rheumatic fever, endocarditis, and other conditions that cause damage to or scarring of the pulmonary valve. Valve replacement may be required in severe cases to restore cardiac function. Flexible valve endovascular prostheses and various delivery devices have been developed so that the valve can be replaced using minimally invasive techniques.

As another example, balloon angioplasty has been used for the treatment of narrowed and occluded blood vessels. A frequent complication associated after the procedure is restenosis, or vessel re-narrowing. To reduce the incidence of re-narrowing, implantable endovascular devices, such as stents, have been used to maintain the patency of the vessel. To improve device effectiveness, stents may be coated with one or more therapeutic agents providing a mode of localized drug delivery. For example, antithrombotic agents may be used to limit clot formation at or near the implanted device. The stent may also be coated with antiproliferative agents or other compounds to reduce excessive endothelial re-growth. Therapeutic agents provided as coatings on implantable medical devices may limit restenosis and reduce the need for repeated treatments to a certain degree.

In the case of a traditional stent, such as one manufactured from nitinol, the deployed stent remains at the treatment site indefinitely. One shortcoming of a permanently deployed stent relates to the fact that with time, endovascular tissue surrounding the stent proliferates. As a result, intimal hyperplasia and significant restenosis can develop. Another procedure may be required at the treatment site to treat the restenosis. However, it may be complicated by the immobility of the ingrown nature of the stent. As such, the stent may need to be removed during an open surgical procedure. To preclude the need for an open surgical procedure, endovascular devices, such as stents, may be manufactured from biodegradable materials. Depending on the constituent material, the stent can degrade in a controlled fashion leaving the treatment site available should future procedures be required.

One complication that is associated with the proper function of endovascular devices, such as valves and stents, is calcification. Over time, calcium can deposit on the device surface leading to restenosis of the blood vessel (e.g., with a stent) or inefficient blood pumping of the heart (e.g., with a prosthetic valve), possibly leading to myocardial infarction. In addition, calcification may interfere with the delivery of therapeutic agents and/or the proper degradation of a stent.

It would be desirable, therefore, to provide a strategy for inhibiting the calcification of endovascular devices that overcomes the aforementioned and other disadvantages.

### SUMMARY OF THE INVENTION

One aspect of the present invention provides an endovascular device including a body including a surface. At least one protein antagonist of calcification is disposed on a portion of the surface.

Another aspect of the invention provides an endoluminal device comprising a body including a surface. A coating is disposed on a portion of the surface. The coating includes a transforming growth factor beta receptor antagonist.

Another aspect of the invention provides a method of inhibiting calcification of an endoluminal device. The method includes providing a body including a surface, disposing at least one protein antagonist of calcification on a portion of the surface, and deploying the body at a treatment site.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a perspective view of a stent delivery system including an endovascular device, made in accordance with the present invention;

FIG. 2 is a perspective view the stent of FIG. 1 shown in an expanded state; and

FIG. 3 is a flowchart illustrating a method of inhibiting calcification of an endoluminal device, in accordance with the present invention.

### DETAILED DESCRIPTION

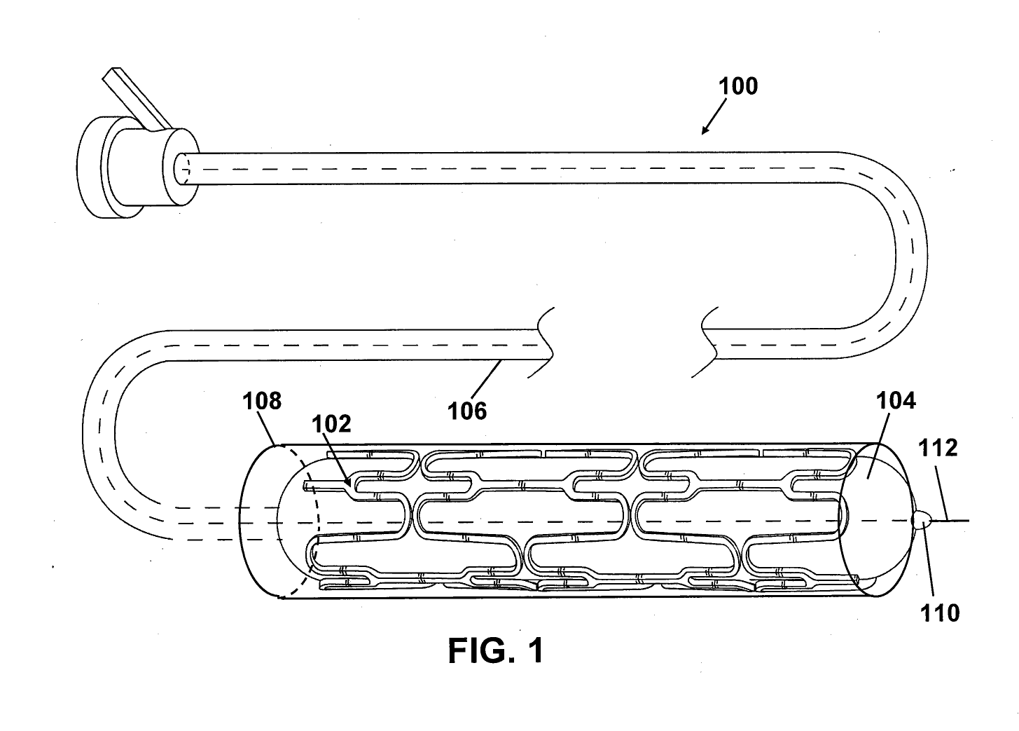

Referring to the drawings, wherein like reference numerals refer to like elements, FIG. 1 is a perspective view of an endovascular system made in accordance with the present invention and shown generally by numeral 100. The endovascular system 100 includes an endovascular device 102. In one embodiment, endovascular device 102 comprises a stent 102. Stent 102 is disposed on a balloon 104 that is operably attached to a catheter 106. Stent 102 (shown in a compressed configuration) remains compressed on balloon 104 during advancement through the vasculature. The compressed stent 102 includes a small profile (i.e., cross-sectional size). In one embodiment, a sheath 108 is disposed on stent 102 to protect stent 102 as well as the vessel walls during advancement.

Although the endovascular device described herein is primarily done so in the context of deployment within a blood vessel, it should be appreciated that endovascular and/or implantable prosthetic devices in accordance with the present invention may be deployed in other vessels, such as a bile duct, intestinal tract, esophagus, and airway. In addition, the nature of the endovascular device can vary from the stent device described herein. In other embodiments, the endovascular device may be, for example, a valve prosthesis, vascular graft, stent-graft, and like devices.

As described herein, the term “biodegradable” refers to one or more substances that degrade (e.g., via hydrolysis) to at least a certain degree within the body. Biodegradable substances are biocompatible and preferably incur a reduced inflammatory response. A “radial” direction is defined as one that is perpendicular to the axis of a vessel blood flow. A “surface” may be the interior, exterior, and/or any side, including any portion of the endoluminal device.

In one embodiment, catheter 106 includes an elongated tubular member manufactured from one or more polymeric materials. In another embodiment, catheter 106 includes a metallic reinforcement element. In some applications (such as smaller, more tortuous vessels), the catheter is constructed from very flexible materials to facilitate advancement into intricate access locations. Numerous over-the-wire, rapid-exchange, and other catheter designs are known and may be adapted for use with the present invention. Catheter 106 can be secured at its proximal end to a suitable Luer fitting, and includes a distal rounded end 110 to reduce harmful contact with a vessel. Catheter 106 can be manufactured from a material such as a thermoplastic elastomer, urethane, polymer, polypropylene, plastic, ethelene chlorotrifluoroethylene (ECTFE), polytetrafluoroethylene (PTFE), fluorinated ethylene propylene copolymer (FEP), nylon, Pebax® resin, Vestamid® nylon, Tecoflex® resin, Halar® resin, Hyflon® resin, Pellathane® resin, combinations thereof, and the like. Catheter 106 includes an aperture formed at the distal rounded end 110 allowing advancement over a guidewire 112.

Balloon 104 may be any variety of balloon or other device capable of expanding stent 102 (e.g., by providing outward radial forces). Balloon 104 may be manufactured from any sufficiently elastic material such as polyethylene, polyethylene terephthalate (PET), nylon, or the like. Those skilled in the art will recognize that stent 102 may be expanded using a variety of means and that the present invention is not limited to balloon expansion.

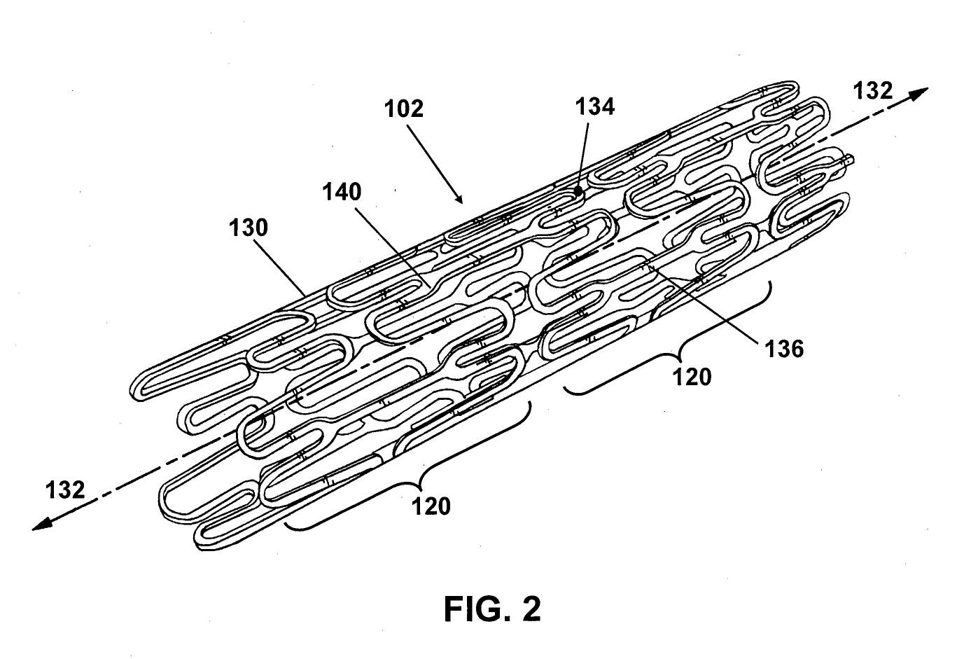

Referring to FIG. 2, in one embodiment, stent 102 may be any variety of implantable prosthetic device having a body with a surface capable of carrying a coating. In one embodiment, stent 102 includes a plurality of identical cylindrical stent segments placed end to end. Two stent segments 120 are shown, and it will be recognized by those skilled in the art that an alternate number of stent segments may be used. The stent 102 includes at least one coating 140 applied to its surface 130. The stent 102 includes a generally tubular body defining a passageway extending along a longitudinal axis 132. The stent 102 is formed from the cylindrical segments 120 arranged successively along longitudinal axis 132. Each of cylindrical segments 120 has a length along longitudinal axis 132 and includes a plurality of roughly W-shaped elements 134. The W-shaped elements 134 open in alternating directions along longitudinal axis 132 about the perimeter or circumference of the cylindrical segments 120. The W-shaped elements 134 are connected to each other by a tie member 136 that is attached to center sections of each of the W-shaped elements 134.

The stent 102 is shown in an expanded state in FIG. 2 in which the cylindrical segments 120 have been expanded radially outward from the longitudinal axis 132. The stent 102 can be compressed into a smaller diameter, as shown in FIG. 1, for delivery within a vessel lumen at which point stent 102 is expanded to provide support to the vessel. In one embodiment, stent 102 may be of the self-expanding variety and manufactured from nickel titanium alloys and other alloys that exhibit superlastic behavior (i.e., capable of significant distortion without plastic deformation). In another embodiment, stent 102 may be designed to be expanded by a balloon or some other device, and may be manufactured from an inert, biocompatible material with high corrosion resistance. The biocompatible material should ideally be plastically deformed at low-moderate stress levels. Suitable materials include, but are not limited to, tantalum, stainless steel, titanium ASTM F63-83 Grade 1, niobium, cobalt-chromium alloys or high carat gold K 19-22. Other suitable materials for the stent 102 include biodegradable compounds such as poly (D,L-lactide/glycolide copolymer), polycaprolactone, poly (hydroxybutyrate-hydroxyvalerate), polyorthoesterpoly-L-lactic acid (PLLA), polyorthoester (POE), natural polymers, and metallic alloys such as those comprising magnesium, and the like, which generally demonstrate high biocompatibility with reduced inflammatory response. Suitable natural polymers include, but are not limited to collagen polymer, polysaccharides, elastin, silk, hyluronic acid, and the like. Such biodegradable materials may additionally be modified to further reduce the inflammatory response as known in the art.

A specific example of a biodegradable stent that may be adapted with the present invention includes the drug-eluting magnesium-based alloy stent that degrades over the course of approximately two months. Those skilled in the art will recognize that a number of non-degradable and/or biodegradable constituent compounds can be used for the manufacture of the stent 102 and are not limited to the example provided herein.

In one embodiment, the stent 102 includes at least one coating 140 applied to a portion of its surface 130. The coating 140 includes a polymer mixed with an inhibition agent. In one embodiment, the inhibition agent comprises at least one osteoinductive compound antagonist. In one embodiment, the at least one osteoinductive compound antagonist comprises a protein or peptide antagonist of a transforming growth factor beta (TGF-β) receptor. More specifically, in one embodiment, the at least one TGF-β receptor antagonist is a bone morphogenic protein (BMP) receptor antagonist. In the same or another embodiment, the at least one TGF-β antagonist is a cytokine, either naturally occurring or genetically modified. In the same or another embodiment, the at least one TGF-β antagonist is a growth differentiation factor (GDF) receptor antagonist. The TGF-β sub-family consists of over 30 structurally related proteins including subfamilies such as BMPs, GDFs, activins, and inhibins, along with more distantly related members such as Nodal and Müllerian Inhibiting Substance (MIS). These ligands are synthesized as prepropeptides of approximately 400-500 amino acids (aa). The N-terminal variable length pro-region is cleaved at a consensus RXXR site prior to secretion. The secreted C-terminal mature segment has 6-7 spatially conserved cysteines that form a cysteine knot structure in the monomer. It is the conserved dimeric structure with two opposing “hands”, however, that give specificity for receptor binding and biological function. Small secondary structural elements arising from the non-conserved regions give family members their specificity for ligand-receptor binding.

In one embodiment, the coating 140 includes at least one of chordin, sclerostin, and/or noggin protein(s), which function to inhibit localized deposition of calcium. In an example, tissue buildup and calcification can be inhibited over an acute inflammatory phase and subsequent endothelial repair of tissue by the inclusion of noggin protein(s) within the coating 140 of the stent 102. In another embodiment, for example, the coating 140 includes one or more proteins which function to inhibit receptors implicated in bone differentiation (e.g., BMP-2, BMP-7 and GDF-5), bone formation (e.g., BMP-3, BMP-3B a.k.a. GDF-10, and BMP-8), and/or bone morphogenesis (e.g., BMP-5 and GDF-3) as understood in the art.

As mentioned above, coating 140 includes a polymer. The polymer provides a matrix for incorporating the inhibition agent within the coating. The coating polymer comprises any suitable biocompatible polymer known in the art. In one embodiment, the biocompatible polymer is biodegradable. Some exemplary biodegradable polymers that may be adapted for use with the present invention include, but are not limited to, collagen polymer, polycaprolactone, polylactide, polyglycolide, polyorthoesters, polyanhydrides, poly(amides), poly(alkyl-2-cyanocrylates), poly(dihydropyrans), poly(acetals), poly(phosphazenes), poly(dioxinones), trimethylene carbonate, polyhydroxybutyrate, polyhydroxyvalerate, their copolymers, blends, and copolymers blends, combinations thereof, and the like. In one embodiment, coating 140 comprises at least one protein antagonist integrated in a natural polymer. Suitable natural polymers include, but are not limited to collagen polymer, polysaccharides, elastin, silk, hyluronic acid, and the like.

In one embodiment, the stent 102 includes at least one therapeutic agent incorporated within the coating 140. The therapeutic agent(s) can be applied to one or more portions of the stent 102. In a biodegradable stent 102, the therapeutic agent may be integrated with the coating 140 thereby allowing elution as the stent 102 degrades. As such, the inhibition of calcification, which may normally interfere with stent degradation, may be reduced.

The therapeutic agent comprises one or more drugs, polymers, a component thereof, a combination thereof, and the like. For example, the therapeutic agent can include a mixture of a drug and a polymer as known in the art. Some exemplary drug classes that may be included are anti-inflammatory agents, antiangiogenesis agents, antiendothelin agents, antimitogenic factors, antioxidants, antiplatelet agents, antiproliferative agents, antisense oligonucleotides, antithrombogenic agents, calcium channel blockers, clot dissolving enzymes, growth factors, growth factor inhibitors, nitrates, nitric oxide releasing agents, vasodilators, virus-mediated gene transfer agents, agents having a desirable therapeutic application, and the like. Specific examples of drugs include abciximab, angiopeptin, colchicine, eptifibatide, heparin, hirudin, lovastatin, methotrexate, rapamycin, streptokinase, taxol, ticlopidine, tissue plasminogen activator, trapidil, urokinase, zotarolimus and growth factors VEGF, IGF, PDGF, and FGF.

In one embodiment, the therapeutic agent polymer provides a matrix for incorporating the drug within the coating, or may provide means for slowing the elution of an underlying therapeutic agent when it comprises a cap coat. The therapeutic agent polymer may be the same as or similar to those described above for the coating polymer.

Solvents are used to dissolve the therapeutic agent and polymer to comprise a therapeutic agent coating solution. Some exemplary solvents that may be adapted for use with the present invention include, but are not limited to, acetone, ethyl acetate, tetrahydrofuran (THF), chloroform, N-methylpyrrolidone (NMP), methylene chloride, and the like.

Those skilled in the art will recognize that the nature of the drug and polymer may vary greatly and are typically formulated to achieve a given therapeutic effect, such as limiting restenosis, thrombus formation, hyperplasia, etc.

In one embodiment, two or more therapeutic agents are incorporated into the stent and are released having a multiple elution profile. For example, a first therapeutic agent disposed on a first portion of the stent 102 may be released to reduce inflammation. The first agent may be released on a short-term basis to overcome surgical trauma of the treatment. Over time, a second therapeutic agent may be eluted at a slower rate to reduce intimal hyperplasia and/or calcification.

The coating 140, with or without additional therapeutic agent(s), may be applied to the surface 130 of the stent 102 by any of numerous strategies known in the art including, but not limited to, spraying, dipping, rolling, nozzle injection, and the like.

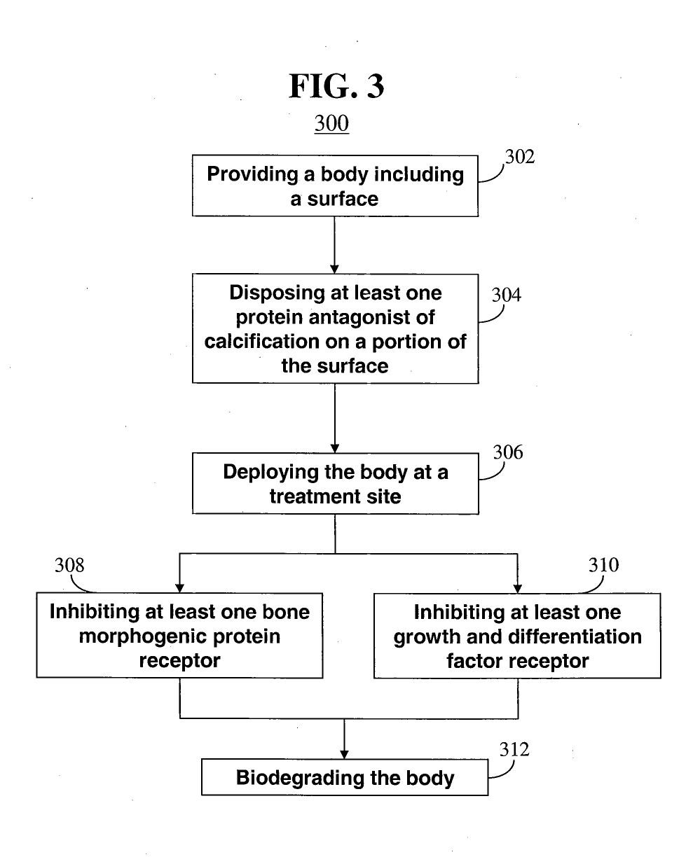

FIG. 3 is a flowchart illustrating method 300 as one embodiment of inhibiting calcification of an endoluminal device, in accordance with the present invention. A stent 102 including a surface 130 is provided, as indicated in Block 302. A coating 140 with at least one protein or peptide antagonist of calcification is disposed on a portion of the surface 130 in a manner as described above. The stent 102 is advanced endovascularly within the patient and deployed at a treatment site. In one embodiment, the balloon 104 is inflated in an axial direction with the stent 102 against a vessel wall, as indicated by Block 304. The balloon 104 is then deflated and removed along with the catheter 106 from the patient leaving the stent 102 in an expanded state at the treatment site, as indicated by Block 306.

In one embodiment, at least one bone morphogenic protein receptor (BMP-R) is inhibited by one or more substances included in the coating 140 of the stent 102 (Block 308). Examples of BMP-Rs include the proteins chordin, sclerostin, and/or noggin proteins. In another or the same embodiment, at least one growth differentiation factor receptor (GDF-R) is inhibited by one or more substances included in the coating 140 of the stent 102 (Block 310). Those skilled in the art will recognize that the inhibitors of GDF-R are not limited to protein molecules. Substances that can attenuate the function of the GDF-R can be used to inhibit calcification of the stent 102, either locally or systemically.

In one embodiment, the stent 102 biodegrades (Block 312). As the calcification of the stent 102 may interfere with the delivery of therapeutic agent(s) and/or the biodegradation of the stent 102, inhibition of calcification may ensure proper agent delivery and stent 102 degradation.

While the embodiments of the invention disclosed herein are presently considered to be preferred, various changes and modifications can be made without departing from the spirit and scope of the invention. For example, the stent configuration is not limited to any particular stent design. In addition, the coating liquid composition and coating process movement characteristics may be varied considerably while providing a desirable coating. Upon reading the specification and reviewing the drawings hereof, it will become immediately obvious to those skilled in the art that myriad other embodiments of the present invention are possible, and that such embodiments are contemplated and fall within the scope of the presently claimed invention. The scope of the invention is indicated in the appended claims, and all changes that come within the meaning and range of equivalents are intended to be embraced therein.

1. An endovascular device comprising:
a body including a surface; and
at least one protein or peptide antagonist of calcification disposed on a portion of the surface, wherein the at least one protein or peptide antagonist comprises at least one transforming growth factor beta receptor antagonist.

2. (canceled)

3. (canceled)

4. The device of claim 1 wherein the at least one transforming growth factor beta receptor antagonist comprises at least one bone morphogenic protein receptor antagonist.

5. The device of claim 1 wherein the at least one transforming growth factor beta receptor antagonist comprises at least one growth differentiation factor receptor antagonist.

6. The device of claim 4 wherein the at least one bone morphogenic protein receptor antagonist is selected from the group consisting of chordin, sclerostin, and noggin.

7. The device of claim 1 wherein the at least one transforming growth factor beta receptor antagonist comprises a cytokine.

8. The device of claim 1 wherein the body is selected from a group consisting of a stent, a valve prosthesis, a vascular graft, and a stent-graft.

9. The device of claim 1 wherein the body is biodegradable.

10. The device of claim 1 wherein the at least one protein antagonist is integrated in a natural polymer.

11. The device of claim 10 wherein the natural polymer is chosen from the group consisting of collagen polymer, polysaccharides, elastin, silk and hyluronic acid.

12. An endoluminal device comprising:
a body including a surface; and
a coating disposed on a portion of the surface; wherein the coating comprises a transforming growth factor beta receptor antagonist.

13. The device of claim 12 wherein the at least one transforming growth factor beta receptor antagonist comprises at least one bone morphogenic protein receptor antagonist.

14. The device of claim 12 wherein the at least one transforming growth factor beta receptor antagonist comprises at least one growth differentiation factor receptor antagonist.

15. The device of claim 12 wherein the body is selected from a group consisting of a stent, a valve prosthesis, a vascular graft, and a stent-graft.

16. The device of claim 12 wherein the body is biodegradable.

17. The device of claim 12 wherein the at least one protein or peptide antagonist is integrated in a natural polymer.

18. The device of claim 17 wherein the natural polymer is chosen from the group consisting of collagen polymer, polysaccharides, elastin, silk and hyluronic acid.

19. A method of inhibiting calcification of an endoluminal device, the method comprising:
providing a body including a surface;
disposing at least one protein or peptide antagonist of calcification on a portion of the surface; and
deploying the body at a treatment site.

20. The method of claim 19 further comprising inhibiting at least one bone morphogenic protein receptor.

21. The method of claim 19 further comprising inhibiting at least one growth differentiation factor receptor.

22. The method of claim 19 further comprising biodegrading the body.
